(19) United States
(12) Patent Application Publication
(10) Pub. No.: US 2015/0157581 A1
(43) Pub. Date: Jun. 11, 2015

(54) BENZYLAMINE CONTAINING COMPOSITIONS AND METHODS FOR APPETITE SUPPRESSION

(71) Applicant: Zija International, Inc., Lehi, UT (US)

(72) Inventor: Joshua James Plant, Provo, UT (US)

(73) Assignee: Zija International, Inc., Lehi, UT (US)

(21) Appl. No.: 14/623,763

(22) Filed: Feb. 17, 2015

Related U.S. Application Data
(62) Division of application No. 14/182,121, filed on Feb. 17, 2014.
(60) Provisional application No. 61/766,694, filed on Feb. 19, 2013.

(51) Int. Cl. A61K 31/137; A61K 36/752; A61K 31/405; A61K 31/4525

(52) U.S. Cl. CPC A61K 31/137; A61K 31/4525; A61K 36/752; A61K 31/405

(57) ABSTRACT

Herbal compositions and techniques for suppressing appetite and managing weight are described. These compositions and techniques are based at least in part on beneficial results showing that benzylamine can suppress appetite. In at least one embodiment, a composition that includes an effective amount of benzylamine and an effective amount of at least one benzylamine enhancer may be utilized for appetite suppression.

### CROSS-REFERENCE TO RELATED APPLICATIONS

This application is a divisional of co-pending U.S. patent application Ser. No. 14/182,121, filed Feb. 17, 2014, entitled “Benzylamine Containing Compositions and Methods for Appetite Suppression,” which application claims the benefit of U.S. Provisional Patent Application No. 61/766,694, filed on Feb. 19, 2013, both of which are hereby incorporated by reference herein in their entireties, including but not limited to those portions that specifically appear hereinafter, the incorporation by reference being made with the following exception: In the event that any portion of the above-referenced applications is inconsistent with this application, this application supersedes said portion of said above-referenced applications.

### BACKGROUND

It is estimated that more than one-third of adults in the United States are obese, and obesity has been on a steady increase over the last 30 years. The rise in obesity can in a large part be attributed to the increased caloric intake in children and adults. Obesity is understood to be associated with various diseases such as heart disease, stroke, type 2 diabetes, and certain types of cancer. These diseases are considered some of the leading causes of preventable death.

### SUMMARY

This Summary is provided to introduce a selection of concepts in a simplified form that are further described below in the detailed description. This Summary is not intended to identify key features or essential features of the claimed subject matter, nor is it intended to be used to limit the scope of the claimed subject matter.

Herbal compositions and techniques for suppressing appetite, decreasing a subject's food consumption, and/or managing a subject's weight are described. These compositions and techniques are based at least in part on appetite suppression characteristics of Moringine (i.e., benzylamine), an alkaloid which Applicant has determined is found in the root of the plant Moringa Oleifera.

In at least one embodiment, a composition for oral consumption that includes an effective amount of benzylamine to suppress appetite may be provided. Any suitable type of dosage form for administering the effective amount may be utilized.

In at least one embodiment, a composition for oral consumption that includes an effective amount of benzylamine and an effective amount of at least one other chemical ingredient to enhance the appetite suppression effect of the benzylamine may be provided.

In at least one embodiment, a process or method of suppressing appetite in a subject can include the subject oral consuming an effective amount of benzylamine for appetite suppression and an effective amount of at least one benzylamine enhancer. As a result, the subject's food intake can be reduced and/or the subject's weight can be managed.

### BRIEF DESCRIPTION OF THE DRAWINGS

The accompanying drawings illustrate implementations of the concepts conveyed in the present application. Features of the illustrated implementations can be more readily understood by reference to the following description taken in conjunction with the accompanying drawings.

FIG. 1 illustrates the chemical structure of benzylamine.

FIG. 2 illustrates an example method that may be implemented in accordance with at least one embodiment of the disclosure.

### DETAILED DESCRIPTION

### Overview

Herbal compositions and techniques for suppressing appetite, reducing a subject's food intake, and/or managing a subject's weight are described. Suppressing appetite can include decreasing the intensity and/or frequency of a subject's desire for food, potentially thereby reducing the subject's intake of food as a result thereof. Managing weight, in turn, can include producing weight loss in a subject and/or maintaining the subject's weight. For purposes of this disclosure, the term “subject” may refer to an animal subject such as a human (e.g., adult human).

The herbal compositions and techniques described herein are based at least in part on appetite suppression characteristics of Moringine (i.e., benzylamine), an alkaloid, which Applicant has determined through high-performance liquid chromatography (HPLC) analysis to be found in the root of the plant Moringa Oleifera.

In at least one embodiment, a composition for oral consumption that includes an effective amount of benzylamine to suppress appetite may be provided. Any suitable type of dosage form for consuming the effective amount may be utilized. For example, a composition formulation of the effective amount of benzylamine and a pharmaceutically acceptable carrier can be provided in a tablet, capsule, pill, liquid, powder (e.g. powder for solution, suspension, or other type of liquid), or other dosage form suitable for oral consumption (i.e. oral ingestion) by a subject. Oral consumption can include, for example, administering the benzylamine-containing composition orally to a subject and/or the subject self-administering the benzylamine-containing composition orally to themselves.

In at least one embodiment, a composition for oral consumption that includes an effective amount of benzylamine and an effective amount of at least one other chemical ingredient to enhance the appetite suppression effect of the benzylamine may be provided. For purposes of this disclosure, this at least one other chemical ingredient(s) may be referred to as herein as a benzylamine enhancer(s).

One example of a benzylamine enhancer is a chemical compound that enhances the absorption of benzylamine after being orally consumed. Another example is a chemical compound that decreases and/or delays the in vivo break down and inactivation of benzylamine by Monoamine Oxidase-B (MAO-B).

### Moringa Oleifera—Benzylamine

Moringa Oleifera is a species of Moringa, a genus in the flowering plant family of Moringaceae. Moringa Oleifera is a widely-grown tree that has been found to be nutritious for humans, providing a variety of vitamins, minerals, and other nutrients. Moringa Oleifera has also been associated with a wide range of human health benefits, including those associated with digestion, eyesight, mental clarity, fatigue, arthritic-like conditions, antiviral activity, improved glucose tolerance, and overall well-being.

### Benzylamine

Generally speaking, most parts of the Moringa Oleifera tree are edible, with the leaves and seeds often being associated with most of the above-mentioned nutritional and health benefits. However, results have shown that moringine, which Applicant has determined is found in the tree's root, can serve as an appetite suppressant when consumed (i.e. ingested) orally by a subject.

Moringine, which may also be referred to by its synonym benzylamine, is a primary amine. The chemical formula for benzylamine can be represented as C7H9N. The chemical structure of benzylamine can be represented as:

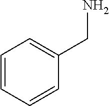

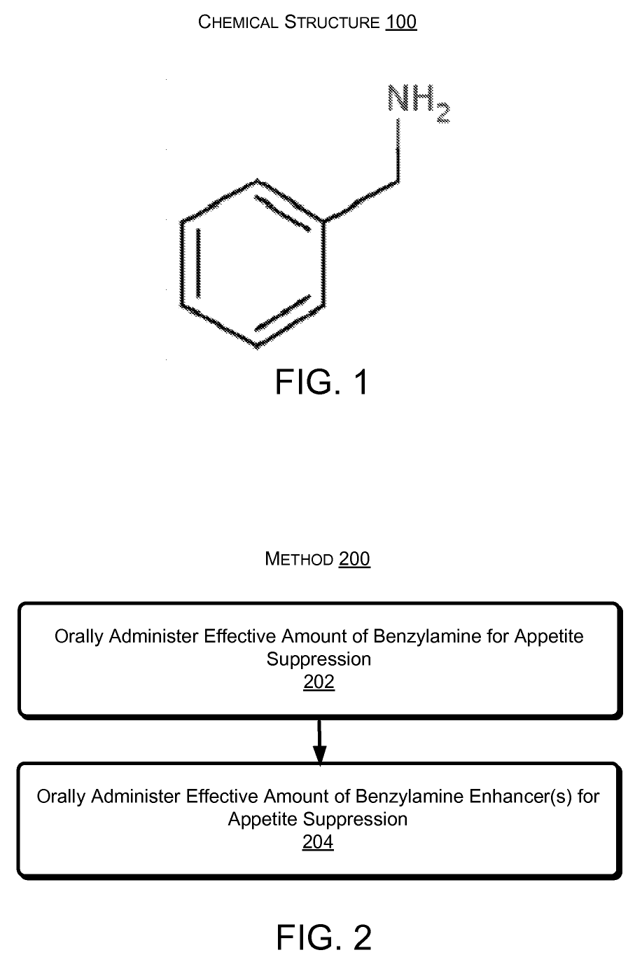

This chemical structure of benzylamine is also illustrated as chemical structure 100 in FIG. 1. Benzylamine's appetite suppression effect may be based at least in part on benzylamine's in vivo potassium-channel blocking properties. More particularly, as a potassium-channel blocker, orally consumed benzylamine can inhibit or mitigate the effects of dopamine in a subject. In other words, once consumed orally by the subject, the benzylamine can produce an in vivo anti-dopaminergic effect in the subject. For example, absorbed benzylamine can result in shorter peak dopamine levels durations (i.e., dopamine spikes) at chemical synapses in the subject, and/or in lower peak dopamine levels at these chemical synapses.

In the context of a subject's appetite, a peak dopamine level might be initiated in the subject when the subject senses (e.g., smells, sees, tastes, etc.) food. However, the absorbed benzylamine might serve to shorten and/or lower this peak, and thus decrease the subject's appetite. In other words, the subject may feel less hungry by virtue of the absorbed benzylamine's in vivo anti-dopaminergic effect. This anti-dopaminergic effect may depend on sufficient levels of benzylamine near dopamine receptors and/or dopamine-producing cells in the subject. These sufficient levels may be sustained for a period of time that corresponds to benzylamine's relatively short in vivo half-life (thought to be approximately 15-30 minutes).

### Benzylamine-Containing Compositions

As noted above, results have shown that Moringine can suppress a subject's appetite when consumed orally by the patient. However, the medium lethal dose (LD50) of benzylamine in subjects is understood to be about 600 milligrams (mg) per kilogram (kg) (i.e., 600 mg/kg). Doses at or near this amount do not appear to be feasible, and would generally be considered unsafe. However, in accordance with the novel appetite suppression compositions and methods described herein, an effective amount of benzylamine that is far below the LD50 can be orally administered to adult humans for appetite suppression.

More particularly, in at least one embodiment, a composition for oral consumption that includes an effective amount of benzylamine to suppress appetite may be provided. Any suitable type of dosage form for administering the effective amount can be utilized. For example, a formulation of the effective amount of benzylamine and a pharmaceutically acceptable carrier may be provided in a tablet, capsule, pill, liquid, powder (e.g. powder for solution, suspension, or other type of liquid), or other dosage form suitable for oral consumption (i.e. oral ingestion) by a subject.

For purposes of this discussion, the term pharmaceutically acceptable carrier may refer to a diluent, adjuvant, excipient, or one or more other pharmacologically inactive and/or inert substances which may be used for manufacturing chemical compositions.

In at least one embodiment, the benzylamine-containing composition may also include an effective amount of at least one other chemical compound—referred to herein as a benzylamine enhancer. A benzylamine enhancer(s) may enhance the benzylamine's appetite suppression effect, and thus decrease the effective amount of administered benzylamine necessary to produce this effect.

One example of a benzylamine enhancer is a chemical compound that may enhance the amount of benzylamine that is absorbed by a subject after being orally consumed by the subject. For purposes of this disclosure, such a compound may be referred to herein as an absorption enhancer. In other words, one example type of benzylamine enhancer is an absorption enhancer which may enhance the amount of orally consumed benzylamine that can be absorbed by a subject. One practical example of an absorption enhancer is piperine (e.g., as found in black pepper), which can be orally consumed with benzylamine.

Another example of a benzylamine enhancer is a chemical compound that decreases and/or delays the in vivo breakdown and inactivation of absorbed benzylamine by Monoamine Oxidase-B (MAO-B). By virtue of its inactivation being decreased and/or delayed, the absorbed benzylamine's anti-dopaminergic effect (and thus appetite suppression effect) may be potentiated, and thus enhanced. For purposes of this disclosure, such a compound may be referred to herein as an activity enhancer. In other words, another example type of benzylamine enhancer is an activity enhancer, which may enhance the anti-dopaminergic effect (and thus appetite suppression effect) of orally consumed benzylamine.

One example of an activity enhancer is a primary amine other than benzylamine that also serves as a target for MAO-B at a chemical synapse. In most or all cases, MAO-B has a lower affinity for benzylamine as compared to other such primary amines.

Consider the primary amine phenylethylamine for instance. At a chemical synapse with concentrations of phenylethylamine and benzylamine, MAO-B is more likely to engage and inactivate phenylethylamine than benzylamine, thus potentiating benzylamine's anti-dopaminergic effect. In other words, phenylethylamine can serve as an activity enhancer by virtue of being a more attractive target at a synapse to MAO-B than benzylamine.

In at least one embodiment, the benzylamine-containing composition may also include an effective amount of at least one other chemical that causes appetite suppression (i.e., an appetite suppressant). In some circumstances, such a chemical may also be considered an absorption enhancer and/or activity enhancer. For example, 5-Hydroxytryptophan (5-HTP) is recognized as an appetite suppressant an activity enhancer that also can decrease and/or delay the in vivo breakdown and inactivation of absorbed benzylamine.

TABLE 1 lists some example benzylamine enhancers (absorption enhancers and activity enhancers), one or more of which may be used either singly or in combination with an effective amount of benzylamine.

TABLE 1
Example Benzylamine Enhancers

| Absorption Enhancers | Activity Enhancers |
|---|---|
| Piperine | Phenylethylamine |
| Grapefruit Extracts (e.g., | Serotonin |
| grapefruit seed extract) | Norepinephrine |
| | Dopamine |
| | 5-Hydroxytryptophan (5-HTP) |

### Benzylamine: Effective Amount and Frequency

As explained above, in accordance with the described techniques, various types of benzylamine-containing compositions for oral consumption may be provided to suppress appetite. In the context of individual oral doses of benzylamine-containing compositions for adult human subjects, the effective amount of orally consumed benzylamine per dose can vary among individuals.

For example, a subject's physiological profile can influence the effect that a certain amount of orally consumed benzylamine will have on a subject's appetite. Alternatively or additionally, the type of dosage form of a composition and/or the ingredients in that dosage form can also influence the effect that this amount will have. For example, benzylamine enhancers can influence the amount of benzylamine that is absorbed into a patient's body after being orally consumed, and/or the benzylamine's anti-dopaminergic effect after being absorbed.

Consistent with the above discussion, an effective amount of benzylamine to be orally consumed by adult human subjects for appetite suppression may be from about 5 mg to about 500 mg per dose (i.e., from about 5-500 mg per dose), with an example effective amount of from about 10 mg to about 150 mg per dose (i.e., from about 10-150 mg per dose).

An example effective amount of benzylamine for oral consumption might be, for instance, about 100 mg per dose. Another higher example effective amount of benzylamine might be about 500 mg per dose.

Note that the above oral doses of benzylamine for appetite suppression in adult human subject are far less than the benzylamine LD50. For example, for a 55 kg (121 pound) human adult consuming 500 mg of benzylamine orally, the mg/kg dosage amount would be approximately 9 mg/kg—far below the benzylamine LD50 of about 600 mg/kg.

Furthermore, an effective dosage frequency (i.e., dosage regimen) for effective amounts of orally consumed benzylamine to adult human subjects for appetite suppression is about 15-30 minutes before meals. This frequency is consistent with what is believed to be the relatively short in vivo half-life of benzylamine (about 15-30 minutes). This dose frequency is also consistent with the explanation that benzylamine's anti-dopaminergic effect can decrease peak dopamine levels, thus suppressing appetite.

### Benzylamine Enhancers: Effective Amounts

As noted above, effective amounts of various types of benzylamine enhancers may be provided in benzylamine-containing compositions that may be orally consumed (e.g. orally administered) for weight suppression.

As with benzylamine, the effective amount of a benzylamine enhancer for human adults can vary among individuals. For example, a subject's physiological profile can influence the effect that a certain amount of an orally consumed benzylamine enhancer will have in increasing the absorption of benzylamine in the subject and/or the anti-dopaminergtic effect that the absorbed benzylamine will have.

Consistent with the above discussion, some effective amounts of some example benzylamine enhancers are provided in TABLE 2 to facilitate the reader's understanding.

TABLE 2
Example Benzylamine Enhancer Effective Amounts

| Benzylamine Enhancers | Effective Amounts (mg/dose) |
|---|---|
| Piperine | From about 1 mg-10 mg, with an |
| | example effective amount of from |
| | about 5-10 mg |
| Grapefruit seed extract | From about 10 mg-200 mg, with an |
| | example effective amount of from |
| | about 75-150 mg |
| Phenylethylamine | From about 50 mg-500 mg, with an |
| | example effective amount of from |
| | about 100-250 mg |
| 5-HTP | From about 20-500 mg, with an |
| | example effective amount of from |
| | about 50-200 mg |

### Example Composition Embodiments

Various embodiments that include a composition with an effective amount of benzylamine for appetite suppression may be provided in accordance with the techniques described herein. TABLES 3 and 4 below each list several non-limiting example embodiments that include an effective amount of benzylamine and at least one other benzylamine enhancer in an oral dosage form, such as a tablet, capsule, pill, liquid, powder (e.g. powder for solution, suspension, or other type of liquid), or other dosage form suitable for oral consumption (i.e. oral ingestion) by a subject.

In this regard, it should be noted that an individual embodiment can include an effective amount of benzylamine per dose and an effective amount of at least one absorption enhancer per dose and/or an effective amount of at least one activity enhancer per dose.

TABLE 3
Example Embodiments

| | Amount of | |
|---|---|---|
| Example | Benzylamine | Amount of Benzylamine Enhancer(s) |
| Embodiment | (mg/dose) | (mg/dose) |
| A* | From about 5- | One or more of the following: |
| | 500 mg | From about 1-10 mg piperine |
| | benzylamine | From about 10-200 mg grapefruit seed |
| | | extract |
| | | From about 50-500 mg |
| | | phenylethylamine |
| | | From about 20-500 mg 5-HTP |
| B* | From about 25- | One or more of the following: |
| | 250 mg | From about 5-10 mg piperine |
| | benzylamine | From about 75-150 mg grapefruit seed |
| | | extract |
| | | From about 100-250 mg |
| | | phenylethylamine |
| | | From about 50-200 mg 5-HTP |
| | From about 10- | One or more of the following: |
| | 150 mg | From about 5-10 mg piperine |
| | benzylamine | From about 75-150 mg grapefruit seed |
| | | extract |
| | | From about 100-250 mg |
| | | phenylethylamine |
| | | From about 50-200 mg 5-HTP |
| E* | From about 50- | One or more of the following: |
| | 250 mg | From about 3-10 mg piperine |
| | benzylamine | From about 75-150 mg grapefruit seed |
| | | extract |
| | | From about 100-250 mg |
| | | phenylethylamine |
| | | From about 50-200 mg 5-HTP |
| F* | From about 50- | One or more of the following: |
| | 125 mg | From about 5-10 mg piperine |
| | benzylamine | From about 100-200 mg grapefruit seed |
| | | extract |
| | | From about 125-250 phenylethylamine |
| | | From about 100-200 mg 5-HTP |
| G* | From about 5 mg | One or more of the following: |
| | benzylamine | From about 5-10 mg piperine |
| | | From about 100-200 mg grapefruit seed |
| | | extract |
| | | From about 250-500 phenylethylamine |
| | | From about 100-200 mg 5-HTP |
| H* | From about | One or more of the following: |
| | 120 mg | From about 5-10 mg piperine |
| | benzylamine | From about 100-200 mg grapefruit seed |
| | | extract |
| | | From about 250-500 phenylethylamine |
| | | From about 100-200 mg 5-HTP |
| I* | From about 25- | One or more of the following: |
| | 125 mg | From about 5-10 mg piperine |
| | benzylamine | From about 100-200 mg grapefruit seed |
| | | extract |
| | | From about 250-500 phenylethylamine |
| | | From about 100-200 mg 5-HTP About 5- |
| | | HTP |
| J* | From about | One or more of the following: |
| | 125 mg | From about 5-10 mg piperine |
| | benzylamine | From about 75-175 mg grapefruit seed |
| | | extract |
| | | From about 250-500 phenylethylamine |
| | | From about 125-250 mg 5-HTP |
| K* | From about 250- | From about 1-10 mg piperine |
| | 500 mg | From about 10-100 mg grapefruit seed |
| | benzylamine | extract |
| | | From about 50-250 mg |
| | | phenylethylamine |
| | | From about 50-100 mg 5-HTP |
| L* | From about | From about 1-10 mg piperine |
| | 250 mg | From about 10-50 mg grapefruit seed |
| | benzylamine | extract |
| | | From about 50-250 mg |
| | | phenylethylamine |
| | | From about 50-100 mg 5-HTP |
| M* | From about | From about 1-10 mg piperine |
| | 500 mg | From about 10-50 mg grapefruit seed |
| | benzylamine | extract |
| | | From about 50-250 mg |
| | | phenylethylamine |
| | | From about 50-100 mg 5-HTP |
| N* | From about 5- | None |
| | 500 mg | |
| | benzylamine | |
| O* | About 10-125 mg | None |
| | benzylamine | |
| P* | About 100- | None |
| | 250 mg | |
| | benzylamine | |
| Q* | About 500 mg | None |
| | benzylamine | |
| R* | About 120 mg | None |
| | benzylamine | |

*Optional: may also include one or more pharmaceutically-acceptable carriers.

TABLE 4
Example Embodiments

Example embodiment 1:
From about 50-100 mg benzylamine
From about 5-10 mg piperine
Optional: one or more pharmaceutically-acceptable carriers
Example embodiment 2:
From about 50-100 mg benzylamine
From about 1-5 mg piperine
Optional: one or more pharmaceutically-acceptable carriers
Example embodiment 3:
From about 100 mg benzylamine
From about 5 mg piperine
Optional: one or more pharmaceutically-acceptable carriers
Example embodiment 4:
From about 100 mg benzylamine
From about 10 mg piperine
Optional: one or more pharmaceutically-acceptable carriers
Example embodiment 5:
From about 250-500 mg benzylamine
From about 10 mg piperine
Optional: one or more pharmaceutically-acceptable carriers
Example embodiment 6:
From about 250 mg benzylamine
From about 5-10 mg piperine
Optional: one or more pharmaceutically-acceptable carriers
Example embodiment 7:
From about 50-100 mg benzylamine
From about 5-10 mg piperine
From about 75-250 mg phenylethylamine
Optional: one or more pharmaceutically-acceptable carriers
Example embodiment 8:
From about 50-100 mg benzylamine
From about 1-5 piperine
From about 125-250 mg phenylethylamine
Optional: one or more pharmaceutically-acceptable carriers
Example embodiment 9:
From about 50-100 mg benzylamine
From about 1-5 piperine
From about 250-500 mg phenylethylamine
Optional: one or more pharmaceutically-acceptable carriers
Example embodiment 10:
From about 50-100 mg benzylamine
From about 5-10 piperine
From about 250-500 mg phenylethylamine
Optional: one or more pharmaceutically-acceptable carriers
Example embodiment 11:
From about 100 mg benzylamine
From about 10 piperine
From about 250 mg phenylethylamine
Optional: one or more pharmaceutically-acceptable carriers
Example embodiment 12:
From about 100 mg benzylamine
From about 5-10 piperine
From about 500 mg phenylethylamine
Optional: one or more pharmaceutically-acceptable carriers
Example embodiment 13:
From about 250 mg benzylamine
From about 10 piperine
From about 500 mg phenylethylamine
Optional: one or more pharmaceutically-acceptable carriers
Example embodiment 14:
From about 250-500 mg benzylamine
From about 10 piperine
From about 250-500 mg phenylethylamine
Optional: one or more pharmaceutically-acceptable carriers
Example embodiment 15:
From about 500 mg benzylamine
From about 10 piperine
From about 250-500 mg phenylethylamine
Optional: one or more pharmaceutically-acceptable carriers
Example embodiment 16:
From about 25-100 mg benzylamine
From about 5-10 mg piperine
From about 50-100 mg grapefruit seed extract
Optional: one or more pharmaceutically-acceptable carriers
Example embodiment 17:
From about 25-75 mg benzylamine
From about 1-5 mg piperine
From about 50-100 mg grapefruit seed extract
Optional: one or more pharmaceutically-acceptable carriers
Example embodiment 18:
From about 50-100 mg benzylamine
From about 5-10 mg piperine
From about 100-200 mg grapefruit seed extract
Optional: one or more pharmaceutically-acceptable carriers
Example embodiment 19:
From about 25-50 mg benzylamine
From about 1-5 mg piperine
From about 100-200 mg grapefruit seed extract
Optional: one or more pharmaceutically-acceptable carriers
Example embodiment 20:
From about 50-100 mg benzylamine
From about 10 mg piperine
From about 100 mg grapefruit seed extract
Optional: one or more pharmaceutically-acceptable carriers
Example embodiment 21:
From about 50-100 mg benzylamine
From about 50-100 mg grapefruit seed extract
Optional: one or more pharmaceutically-acceptable carriers
Example embodiment 22:
From about 250 mg benzylamine
From about 50-100 mg grapefruit seed extract
Optional: one or more pharmaceutically-acceptable carriers
Example embodiment 23:
From about 250-250 mg benzylamine
From about 50-100 mg grapefruit seed extract
Optional: one or more pharmaceutically-acceptable carriers
Example embodiment 24:
From about 100 mg benzylamine
From about 50-100 mg grapefruit seed extract
Optional: one or more pharmaceutically-acceptable carriers
Example embodiment 25:
From about 25-100 mg benzylamine
From about 50-100 mg grapefruit seed extract
From about 100-200 mg phenylethylamine
Optional: one or more pharmaceutically-acceptable carriers
Example embodiment 26:
From about 50-100 mg benzylamine
From about 100-150 mg grapefruit seed extract
From about 250-500 mg phenylethylamine
Optional: one or more pharmaceutically-acceptable carriers
Example embodiment 27:
From about 100 mg benzylamine
From about 100 mg grapefruit seed extract
From about 500 mg phenylethylamine
Optional: one or more pharmaceutically-acceptable carriers
Example embodiment 28:
From about 250 mg benzylamine
From about 200 mg grapefruit seed extract
From about 250 mg phenylethylamine
Optional: one or more pharmaceutically-acceptable carriers
Example embodiment 29:
From about 250 mg benzylamine
From about 200 mg grapefruit seed extract
From about 250 mg phenylethylamine
From about 5-10 mg piperine
Optional: one or more pharmaceutically-acceptable carriers
Example embodiment 30:
From about 50-100 mg benzylamine
From about 100 mg phenylethylamine
Optional: one or more pharmaceutically-acceptable carriers
Example embodiment 31:
From about 100-250 mg benzylamine
From about 10 mg piperine
Optional: one or more pharmaceutically-acceptable carriers
Example embodiment 32:
From about 100 mg benzylamine
From about 1-5 mg of piperine
Optional: one or more pharmaceutically-acceptable carriers
Example embodiment 33:
From about 100 mg benzylamine
From about 10 mg piperine
From about 250 mg phenylethylamine
Optional: one or more pharmaceutically-acceptable carriers
Example embodiment 34:
From about 50-100 mg benzylamine
From about 50-125 mg grapefruit seed extract
Optional: one or more pharmaceutically-acceptable carriers
Example embodiment 35:
From about 100 mg benzylamine
From about 250 mg grapefruit seed extract
From about 250-500 mg phenylethylamine
Optional: one or more pharmaceutically-acceptable carriers
Example embodiment 36:
From about 150-250 mg benzylamine
From about 250 mg phenylethylamine
Optional: one or more pharmaceutically-acceptable carriers
Example embodiment 37:
From about 50-100 mg benzylamine
From about 50-200 mg 5-HTP
Optional: one or more pharmaceutically-acceptable carriers
Example embodiment 38:
From about 50-100 mg benzylamine
From about 50-100 mg 5-HTP
Optional: one or more pharmaceutically-acceptable carriers
Example embodiment 39:
From about 100 mg benzylamine
From about 100-200 mg 5-HTP
Optional: one or more pharmaceutically-acceptable carriers
Example embodiment 40:
From about 100 mg benzylamine
From about 200 mg 5-HTP
Optional: one or more pharmaceutically-acceptable carriers
Example embodiment 41:
From about 50-100 mg benzylamine
From about 5-10 mg of piperine
From about 50-200 mg 5-HTP
Optional: one or more pharmaceutically-acceptable carriers
Example embodiment 42:
From about 50-100 mg benzylamine
From about 1-5 mg piperine
From about 50-100 mg 5-HTP
Optional: one or more pharmaceutically-acceptable carriers
Example embodiment 43:
From about 100 mg benzylamine
From about 5 mg piperine
From about 100-200 mg 5-HTP
Optional: one or more pharmaceutically-acceptable carriers
Example embodiment 44:
From about 100 mg benzylamine
From about 10 mg piperine
From about 200 mg 5-HTP
Optional: one or more pharmaceutically-acceptable carriers
Example embodiment 45:
From about 250-500 mg benzylamine
From about 10 mg piperine
From about 100 mg 5-HTP
Optional: one or more pharmaceutically-acceptable carriers
Example embodiment 46:
From about 50-100 mg benzylamine
From about 50-200 mg 5-HTP
From about 75-250 mg phenylethylamine
Optional: one or more pharmaceutically-acceptable carriers
Example embodiment 47:
From about 50-100 mg benzylamine
From about 100-200 mg 5-HTP
From about 75-250 mg phenylethylamine
Optional: one or more pharmaceutically-acceptable carriers
Example embodiment 48:
From about 50-100 mg benzylamine
From about 1-5 piperine
From about 125-250 mg phenylethylamine
From about 100-200 mg 5-HTP
Optional: one or more pharmaceutically-acceptable carriers
Example embodiment 49:
From about 50-100 mg benzylamine
From about 1-5 piperine
From about 250-500 mg phenylethylamine
From about 200 mg 5-HTP
Optional: one or more pharmaceutically-acceptable carriers
Example embodiment 50:
From about 50-100 mg benzylamine
From about 5-10 piperine
From about 250-500 mg phenylethylamine
From about 100 mg 5-HTP
Optional: one or more pharmaceutically-acceptable carriers
Example embodiment 51:
From about 25-100 mg benzylamine
From about 5-10 mg piperine
From about 50-100 mg grapefruit seed extract
From about 50-200 mg 5-HTP
Optional: one or more pharmaceutically-acceptable carriers
Example embodiment 52:
From about 25-75 mg benzylamine
From about 50-200 mg 5-HTP
From about 50-100 mg grapefruit seed extract
Optional: one or more pharmaceutically-acceptable carriers
Example embodiment 53:
From about 50-100 mg benzylamine
From about 100-200 mg 5-HTP
From about 100-200 mg grapefruit seed extract
Optional: one or more pharmaceutically-acceptable carriers
Example embodiment 54:
From about 25-50 mg benzylamine
From about 1-5 mg piperine
From about 100-200 mg grapefruit seed extract
From about 200 mg 5-HTP
Optional: one or more pharmaceutically-acceptable carriers
Example embodiment 55:
From about 50-100 mg benzylamine
From about 10 mg piperine
From about 100 mg grapefruit seed extract
From about 50-200 mg 5-HTP
Optional: one or more pharmaceutically-acceptable carriers
Example embodiment 56:
From about 50-100 mg benzylamine
From about 50-100 mg grapefruit seed extract
From about 200 mg 5-HTP
Optional: one or more pharmaceutically-acceptable carriers
Example embodiment 57:
From about 250 mg benzylamine
From about 50-100 mg grapefruit seed extract
Optional: one or more pharmaceutically-acceptable carriers
Example embodiment 58:
From about 200 mg 5-HTP
From about 50-100 mg grapefruit seed extract
Optional: one or more pharmaceutically-acceptable carriers
Example embodiment 59:
From about 100 mg benzylamine
From about 50-100 mg grapefruit seed extract
Optional: one or more pharmaceutically-acceptable carriers
Example embodiment 60:
From about 25-100 mg benzylamine
From about 50-100 mg grapefruit seed extract
From about 100-200 mg phenylethylamine
From about 50-200 mg 5-HTP
Optional: one or more pharmaceutically-acceptable carriers

### Example Method

To assist the reader in understanding the described techniques for suppressing appetite and managing weight, FIG. 2 illustrates an example process or method of suppressing appetite in a subject. As a result, the subject's food intake can be reduced and/or the subject's weight can be managed.

Regarding the method 200 illustrated in FIG. 2, at block 202 an effective amount (i.e., dose) of benzylamine can be orally consumed by a subject for appetite suppression. For example, an effective amount of benzylamine might be administered orally to, or self-administered orally by, an adult human. As explained above, any suitable type of dosage form for oral consumption can be utilized. For example, a formulation of the effective amount of benzylamine and a pharmaceutically acceptable carrier may be provided in a tablet, capsule, pill, liquid, powder (e.g. powder for solution, suspension, or other type of liquid), or other dosage form suitable for oral consumption (i.e. oral ingestion) by a subject.

At Block 204, and effective amount of at least one benzylamine enhancer may be orally consumed by the subject for appetite suppression. For example, an effective amount of at least one absorption enhancer and/or an effective amount of at least one activity enhancer might be orally administered to, or orally self-administered by, the adult human in a suitable dosage form (e.g., tablet, capsule, pill, liquid, powder, etc. for oral consumption).

In at least one embodiment, the effective amount of the at least one benzylamine enhancer and the at least one benzylamine enhancer may be orally consumed by the subject together in the same dosage form. In other words, the effective amount of the at least one absorption enhancer and/or the effective amount of the activity enhancer may be provided with the effective amount of the benzylamine at block 202 in the same suitable dosage form e.g., tablet, capsule, pill, liquid, etc. for oral consumption). Without limitation, some example embodiments that include an effective amount of benzylamine and at least one benzylamine enhancers are listed in TABLES 3 and 4 above.

In the foregoing Detailed Description of the Disclosure, various features of the disclosure are grouped together in a single embodiment for the purpose of streamlining the disclosure. This method of disclosure is not to be interpreted as reflecting an intention that the claimed disclosure requires more features than are expressly recited in each claim. Rather, inventive aspects lie in less than all features of a single foregoing disclosed embodiment. Those skilled in the art will recognize different variations and modifications that can be made to compositions and method in accordance with the disclosure without departing from the scope or spirit of the disclosure.

It is to be understood that the above-described arrangements are only illustrative of the application of the principles of the disclosure. Numerous modifications and alternative arrangements may be devised by those skilled in the art without departing from the spirit and scope of the disclosure. Thus, while the disclosure has been described above with particularity and detail, it will be apparent to those skilled in the art that numerous modifications, including, but not limited to, variations in amount, ingredients, form, function and manner of operation, assembly and use may be made without departing from the principles and concepts set forth herein.

Certain terms are used throughout the description to refer to particular system components. As one skilled in the art will appreciate, components may be referred to by different names. This document does not intend to distinguish between components that differ in name, but not function.

The foregoing description has been presented for the purposes of illustration and description. It is not intended to be exhaustive or to limit the disclosure to the precise form disclosed. Many modifications and variations are possible in light of the above teaching. Further, it should be noted that any or all of the aforementioned alternate implementations may be used in any combination desired to form additional hybrid implementations of the disclosure.

1-16. (canceled)

17. A method of suppressing appetite in a subject comprising orally consuming an effective amount of benzylamine for appetite suppression and an effective amount of at least one benzylamine enhancer.

18. The method of claim 17, wherein the effective amount of benzylamine comprises about 5 to 500 milligrams benzylamine.

19. The method of claim 17, wherein the benzylamine enhancer comprises one or more of:
piperine;
grapefruit seed extract;
Phenylethylamine; or
Hydroxytryptophan (5-HTP).

20. The method of claim 17, wherein the effective amount of at least one benzylamine enhancer comprises one or more of:
from about 1 to 10 milligrams of piperine;
from about 10 to 200 milligrams of grapefruit seed extract;
from about 50 to 500 milligrams of Phenylethylamine; or
from about 20 to 500 milligrams of Hydroxytryptophan (5-HTP).

21. The method of claim 18, wherein the effective amount of benzylamine comprises from about 10 to 150 milligrams benzylamine.

22. The method of claim 17, wherein the composition is in a form suitable for oral consumption.

23. The method of claim 17, wherein the composition further comprises an effective amount of one or both of: at least one absorption enhancer or at least one activity enhancer.

24. The method of claim 23, wherein the at least one absorption enhancer comprises an effective amount of at least one of: piperine or grapefruit seed extract.

25. The method of claim 23, wherein the at least one activity enhancer comprises an effective amount of at least one of: phenylethylamine or Hydroxytryptophan (5-HTP).

26. The method of claim 17, wherein the effective amount of at least one benzylamine enhancer comprises one or both of:
from about 1 to 10 milligrams of piperine; or
from about 10 to 200 milligrams of grapefruit seed extract.

27. The method of claim 26, wherein the effective amount of at least one benzylamine enhancer comprises one or both of:
from about 5-10 milligrams of piperine; or
from about 75-150 milligrams of grapefruit seed extract.

28. The method of claim 17, wherein the effective amount of at least one benzylamine enhancer comprises one or both of:
from about 50 to 500 milligrams of Phenylethylamine; or
from about 20 to 500 milligrams of Hydroxytryptophan (5-HTP).

29. The method of claim 28, wherein the effective amount of at least one benzylamine enhancer comprises one or both of:
from about 100 to 250 milligrams of Phenylethylamine; or
from about 50 to 200 milligrams of 5-HTP.

30. A method of suppressing appetite in a subject comprising orally consuming an effective amount of benzylamine for appetite suppression.

31. The method of claim 30, wherein the effective amount of benzylamine comprises from about 5 to 500 milligrams benzylamine.

32. The method of claim 31, wherein the effective amount of benzylamine comprises from about 10 to 150 milligrams benzylamine.

33. The method of claim 30, wherein the composition is in a form suitable for oral consumption.

34. The method of claim 30, wherein the composition further comprises an effective amount of one or both of: at least one absorption enhancer or at least one activity enhancer.

35. The method of claim 34, wherein the at least one absorption enhancer comprises an effective amount of at least one of: piperine or grapefruit seed extract.

36. The method of claim 34, wherein the at least one activity enhancer comprises an effective amount of at least one of: phenylethylamine or Hydroxytryptophan (5-HTP).
